(19) United States
(12) Patent Application Publication
(10) Pub. No.: US 2010/0280133 A1
(43) Pub. Date: Nov. 4, 2010

(54) AQUEOUS EMULSION CONTAINING A CYCLODEXTRIN DERIVATIVE, A PERFUME, AND A POLYSORBATE

(75) Inventors: Maria-Elisabeth Regiert, Munich (DE); Michaela Kupka, Burghausen (DE); Harald Sigl, Toeging (DE)

Correspondence Address: BROOKS KUSHMAN P.C., 1000 TOWN CENTER, TWENTY-SECOND FLOOR, SOUTHFIELD, MI, 48075, US

(73) Assignee: WACKER CHEMIE AG, Munich (DE)

(21) Appl. No.: 12/811,941

(22) PCT Filed: Jan. 9, 2009
(86) PCT No.: PCT/EP09/50196

§ 371 Date: Jul. 7, 2010

(30) Foreign Application Priority Data
Jan. 17, 2008 (DE) 10 2008 000 084.1

(51) Int. Cl. A01N 25/04; A01N 25/00; A01P 3/00; A61P 31/00; A61Q 13/00

(52) U.S. Cl. 514/777; 512/12; 106/205.1

(57) ABSTRACT

Aqueous fragrance emulsions are prepared from a hydrophobic fragrance substance, a cyclodextrin derivative, and a polysorbate. The emulsions are stable despite containing a high proportion of fragrance and can be used to prepare uniform coatings on substrates.

The invention relates to an emulsion comprising a cyclodextrin derivative, a odorous substance and a polysorbate, and methods for preparing this emulsion and its use.

Cyclodextrin derivatives are chemically modified cyclodextrins. Cyclodextrins are cyclic oligosaccharides which are composed of 6, 7 or 8 α(1-4)-linked anhydroglucose units. The α-, β- or γ-cyclodextrins produced by enzymatic starch conversion differ in the diameter of their hydrophobic cavity and are suitable for the inclusion of numerous lipophilic substances. Cyclodextrin derivatives have a considerably higher solubility in water than the corresponding unmodified cyclodextrin. Cyclodextrin derivatives can be used for modifying the properties of other compounds, for example for increasing the water solubility of oil-soluble substances.

Odorous substances is the term used to refer to all natural and synthetic substances which have an odor. They are often readily volatile and unstable toward oxidizing agents. Consequently, even in air and in light, the behavior of these products often changes. Examples of odorous substances of low volatility, i.e. substances with a high boiling point, are benzophenone, benzyl salicylate, galaxolides, derivatives of cinnamaldehyde, or phenylethyl phenylacetate. Examples of volatile odorous substances are cinnamyl alcohol, coumarin, derivatives of salicylic acid, phenolic odorous substances such as vanillin or eugenol and cedryl derivatives. Examples of highly volatile odorous substances are aldehydes, alcohols, and esters of terpenes such as citral, linalool or geranyl acetate. Odorous substances are also essential oils such as tea tree oil, citrus oils (lemon oil, orange oil), oils from herbs (rosemary oil, thyme oil, oregano oil), oils from woods (rosewood oil, cedarwood oil) cinnamaldehydes** or cinnamon bark oil, eugenol** or clove flower oil, 4-terpineol, terpineols** or tea tree oil*, thymol** or thyme oil*, cineol** or eucalyptus oil, camphor** or camphor tree oil, geraniol** or palmarosa oil, citronella oil and geranium oil*, cedrol**, cedarwood oil*. The substances marked with ** are active components and in most cases main components of the specified essential oils marked with *. Based on the effect of individual components, these oils can have an antimicrobial, fungicidal and, as a result of their odor, insect-repelling effect.

Odorous substances are generally lipophilic and therefore form separate phases even when added in small amounts to an aqueous phase, which limits the broad use, desired per se, of odorous substances in purely aqueous preparations such as coating compositions. It has therefore already been attempted to increase the water solubility of the odorous substances through complexation with cyclodextrins or cyclodextrin derivatives.

In this respect, EP 1 002 549 A1 discloses a clear aqueous formulation which comprises 0.01-1% by weight of a perfume in an aqueous cyclodextrin solution comprising 0.1-5% by weight of a cyclodextrin.

WO 96/04938 describes a clear aqueous composition for odor reduction comprising 0.01-1% by weight of a perfume, and up to 5% by weight of water-soluble cyclodextrins.

Products of this type are used for the coating of surfaces, as disclosed in the aforementioned specifications.

The prior art thus describes stable compositions comprising up to 1% by weight of perfume in a clear aqueous cyclodextrin preparation. In the event of a further addition of perfume to the clear aqueous cyclodextrin preparation, the latter is not stable, i.e. two phases are formed within minutes. Such unstable aqueous cyclodextrin preparations with lipophilic, i.e. oil-soluble, odorous substances are unsuitable for the coating of surfaces.

It was an object of the invention to provide a stable aqueous, cyclodextrin preparation which comprises larger amounts of hydrophobic odorous substances than the preparations of the prior art.

This object is achieved by an aqueous emulsion comprising a cyclodextrin derivative, a hydrophobic odorous substance and a polysorbate, characterized in that it comprises the cyclodextrin derivative in an amount of from 6 to 35% by weight, preferably 10 to 20% by weight, and the hydrophobic odorous substance in an amount of from 3 to 20% by weight, preferably 6 to 12% by weight, and the polysorbate in an amount of from 4 to 20% by weight, preferably 6 to 12% by weight, and water.

The hydrophobic odorous substance is preferably a perfume, particularly preferably a hydrophobic odorous substance which has an antimicrobial, fungicidal or, as a result of its odor, insect-repelling effect.

The cyclodextrin derivative is preferably a derivative of α-, β- or γ-cyclodextrin, it is particularly preferably methyl β-cyclodextrin (M-beta-CD) or hydroxypropyl beta-cyclodextrin (HP-beta-CD).

The polysorbate is perferably polyoxyethylene(20) sorbitan monooleate (polysorbate 80), commercially available under the name Tween® 680 from Lonza Basel, Switzerland, polyoxyethylene sorbitan tristearate (polysorbate 65), commercially available under the name Tween® 65 from Lonza Basel, Switzerland or polyoxyethylene(20) sorbitan monolaurate (polysorbate 20), commercially available under the name Tween® 20 from Lonza Basel, Switzerland.

It is particularly preferably Tween® 20 (polyoxyethylene(20) sorbitan monolaurate).

Besides water, the emulsion particularly preferably comprises 6 to 35% by weight of methyl beta-cyclodextrin, or hydroxypropyl beta-cyclodextrin, 3 to 20% by weight of a odorous substance and 4 to 20% by weight of polyoxyethylene(20) sorbitan monolaurate.

Within the context of the present invention, stable is understood as meaning that the emulsion exhibits no phase separation over a period of at least 2 weeks, preferably more than 20 weeks.

The emulsion according to the invention is more effective than the clear one described in the prior art. The release of the guest substance, for example of the odorous substance can be detected more strongly by sensory means (odor strength 0-15) and also by analytical means (via solid phase microextraction/SPME analysis).

The emulsion according to the invention can be prepared in a manner known per se. This can happen, for example, by processing a mixture of 6 to 35% by weight, preferably 10 to 20% by weight, of cyclodextrin derivative and 3 to 20% by weight, preferably 6 to 12% by weight, of hydrophobic odorous substance and 4 to 20% by weight, preferably 6 to 12% by weight, of polysorbate in water to give a stable emulsion. The mixture of said components is stirred intensively for this.

The invention thus also relates to a method for preparing an emulsion, which is characterized in that a mixture of 6 to 35% by weight, preferably 10 to 20% by weight, of cyclodextrin or cyclodextrin derivative and 3 to 20% by weight, preferably 6 to 12% by weight, of hydrophobic odorous substance and 4 to 20% by weight, preferably 6 to 12% by weight, of polysorbate is processed in water to give a stable emulsion.

The emulsion according to the invention is suitable in particular for the coating of surfaces since the emulsion according to the invention distributes extraordinarily evenly on any surfaces and penetrates into these only to a moderate degree. Coating of the surfaces with the emulsion according to the invention takes place without streaks and mottling. A double coating is superfluous in most cases, the coated surfaces are more even than surfaces coated with clear aqueous cyclodextrin preparations. No colored edges are formed.

For this, the emulsion according to the invention can be applied directly to the surface to be coated, although it can firstly also be introduced into a coating composition. The coating compositions may be a paint, e.g. a wall paint. Preferably, the emulsion is incorporated into the coating composition in an amount of from 0.1 to 90% by weight.

The invention thus also relates to a coating composition comprising an emulsion according to the invention.

The surfaces to be coated are, for example, mineral or maximum content in a stable aqueous solution of the corresponding cyclodextrin derivative (HP-beta CD). The preparation of the respective compositions is described in examples 8 to 10.

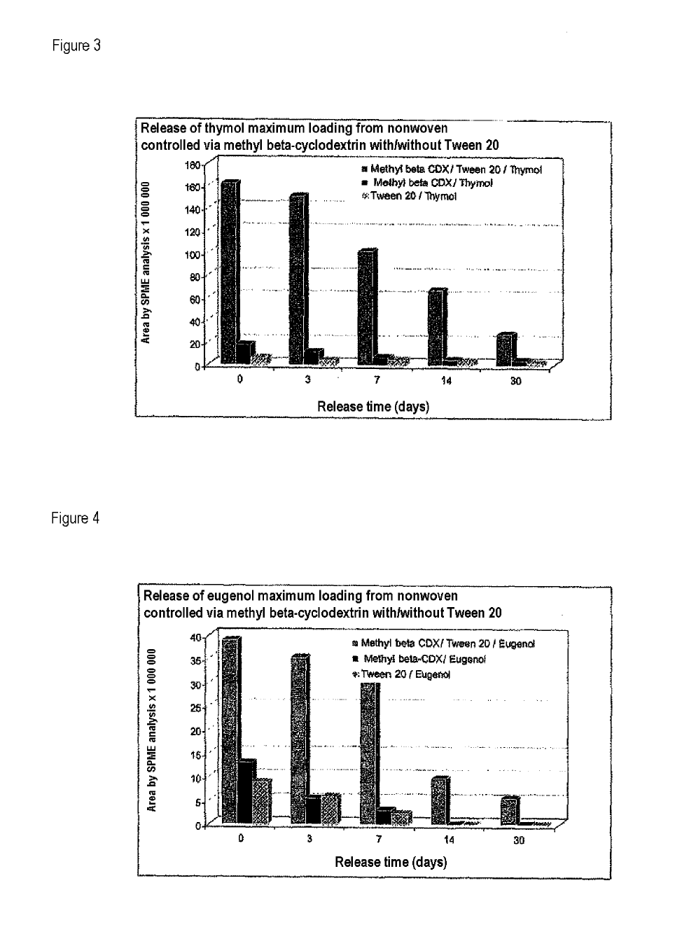

FIG. 3 shows the release of thymol from a piece of nonwoven as described in example 11.

FIG. 4 shows the release of eugenol from a piece of nonwoven as described in example 12.

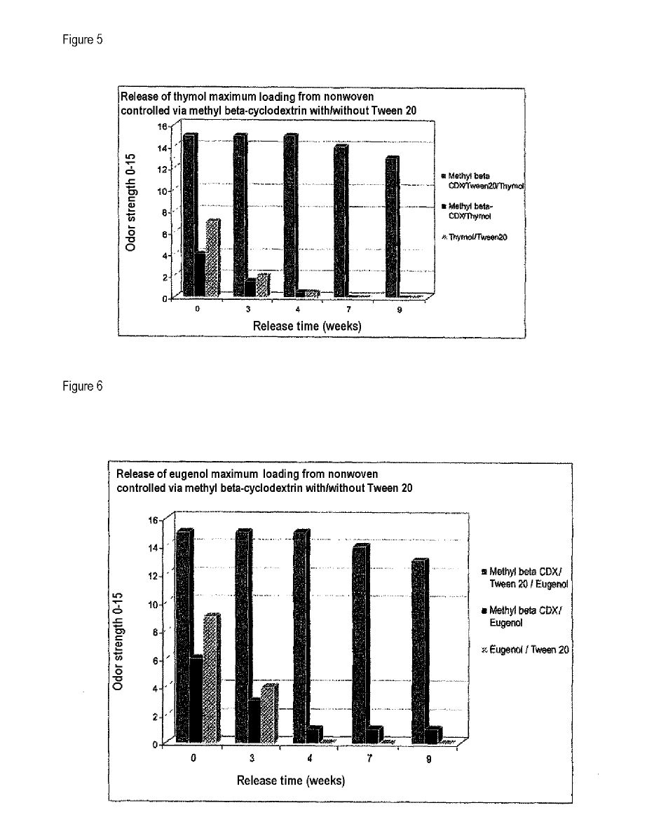

FIG. 5 shows the release of thymol from a piece of nonwoven as described in example 11.

FIG. 6 shows the release of eugenol from a piece of nonwoven as described in example 12.

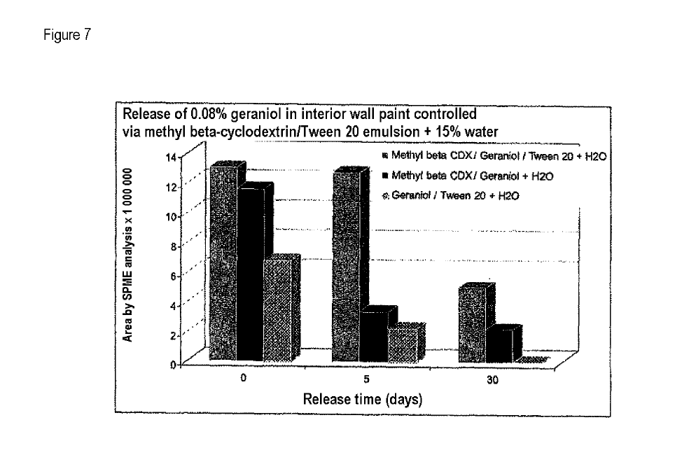

FIG. 7 shows the release of geraniol from a piece of nonwoven as described in example 13.

The examples below serve to further illustrate the invention. In the examples, in each case the preparation of a stable aqueous emulsion comprising the odorous substance specified in each case is described. In addition, the maximum amount of odorous substance at which a stable emulsion is still obtained is described. a) and, if appropriate, c) each describes the prior art and b) the composition according to the invention.

### EXAMPLE 1a

0.4 g of cinnamaldehyde or cinnamon bark oil is dissolved in 34.6 g of a 10% strength (w/w) aqueous solution of methyl beta-cyclodextrin by stirring.

### EXAMPLE 1b

4.2 g of Tween 20® and 4.2 g of cinnamaldehyde or cinnamon bark oil are emulsified in 26.6 g of a 10% strength aqueous solution of methyl beta-cyclodextrin organic surfaces. Examples of mineral surfaces are stone, concrete or construction materials. Examples of organic surfaces are inanimate organic surfaces such as textiles/nonwoven, wood, paper or leather, but also living organic surfaces such as skin or hair.

The coating of the surfaces takes place here by applying the emulsion according to the invention or the coating composition according to the invention and, where necessary, drying the surface.

A surface coated with an emulsion according to the invention has various advantages: in the case of inanimate organic surfaces such as nonwovens or textiles, the nature (“hand”) of the material is not changed. In contrast to this, the coating of the surface with a clear aqueous cyclodextrin preparation without the addition of polysorbates with an identical active ingredient concentration of the odorous substance leads to rigidity of the coated fabric, the wearing property of which is described as sticky.

The invention thus also relates to surfaces which are characterized in that they have been coated with an emulsion according to the invention or a coating composition according to the invention.

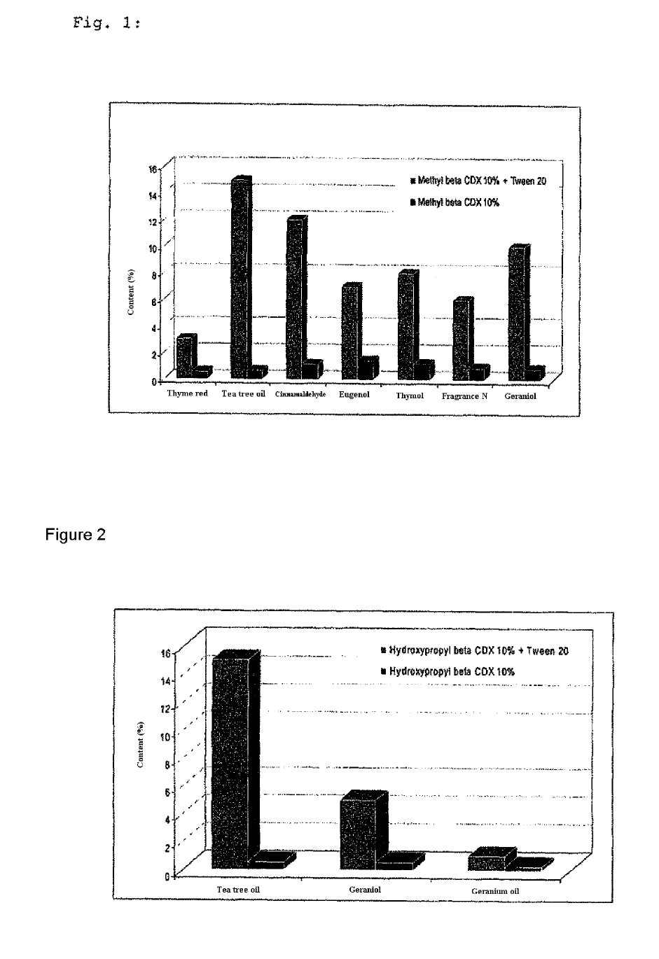

FIG. 1 shows the maximum content of the odorous substance specified in each case in a stable aqueous emulsion according to the invention compared to the maximum content in a stable aqueous solution of the corresponding cyclodextrin derivative (M-beta CD). The preparation of the respective compositions is described in examples 1 to 7.

FIG. 2 shows the maximum content of the odorous substance specified in each case in a stable aqueous emulsion according to the invention compared to the by stirring.

### EXAMPLE 2a

0.22 g of tea tree oil is dissolved in 34.78 g of a 10% strength aqueous solution of methyl beta-cyclodextrin by stirring.

### EXAMPLE 2b

5.25 g of Tween 20® and 5.25 g of tea tree oil are emulsified in 24.5 g of a 10% strength aqueous solution of methyl beta-cyclodextrin by stirring.

### EXAMPLE 3a

0.42 g of thymol is dissolved in 34.58 g of a 10% strength aqueous solution of methyl beta-cyclodextrin by stirring.

### EXAMPLE 3b

2.8 g of Tween 20® and 2.8 g of thymol are emulsified in 29.4 g of a 10% strength aqueous solution of methyl beta-cyclodextrin by stirring.

### EXAMPLE 3c

2.8 g of thymol are emulsified in 2.8 g of Tween 20® by stirring.

### EXAMPLE 4a

0.196 g of thyme red oil is dissolved in 34.804 g of a 10% strength aqueous solution of methyl beta-cyclodextrin by stirring.

### EXAMPLE 4b

1.5 g of Tween 20® and 1.5 g of thyme red oil are emulsified in 47 g of a 10% strength aqueous solution of methyl beta-cyclodextrin by stirring.

### EXAMPLE 5a

0.49 g of eugenol is dissolved in 34.51 g of a 10% strength aqueous solution of methyl beta-cyclodextrin by stirring.

### EXAMPLE 5b

2.45 g of Tween 20® and 2.45 g of eugenol are emulsified in 30.0 g of a 10% strength aqueous solution of methyl beta-cyclodextrin by stirring.

### EXAMPLE 5c

2.8 g of eugenol are homogenized in 2.8 g of Tween 20® by stirring.

### EXAMPLE 6a

0.3 g of Fragrance N is dissolved in 34.7 g of a 10% strength aqueous solution of methyl beta-cyclodextrin by stirring.

### EXAMPLE 6b

2.1 g of Tween 20® and 2.1 g of Fragrance N are emulsified in 30.8 g of a 10% strength aqueous solution of methyl beta-cyclodextrin by stirring.

### EXAMPLE 7a

0.28 g of geraniol is dissolved in 34.7 g of a 10% strength aqueous solution of methyl beta-cyclodextrin by stirring.

### EXAMPLE 7b

2.8 g of Tween 20® and 2.8 g of geraniol are emulsified in 22.3 g of a 10% strength aqueous solution of methyl beta-cyclodextrin by stirring.

### EXAMPLE 7c

2.8 g of geraniol are homogenized in 2.8 g of Tween 20® by stirring.

### EXAMPLE 7d: (According to the Invention)

0.28 g of Tween 20® and 0.28 g of geraniol are homogenized in 30.8 g of a 10% strength aqueous solution of methyl beta-cyclodextrin by stirring.

### EXAMPLE 8a

0.5 g of tea tree oil is dissolved in 99.5 g of a 10% strength aqueous solution of hydroxypropyl beta-cyclodextrin by stirring.

### EXAMPLE 8b

15 g of tea tree oil and 15 g of Tween 20® are emulsified in 70 g of a 10% strength aqueous solution of hydroxypropyl beta-cyclodextrin by stirring.

### EXAMPLE 9a

0.5 g of geraniol is homogenized in 99.5 g of a 10% strength aqueous solution of hydroxypropyl beta-cyclodextrin by stirring.

### EXAMPLE 9b

5 g of geraniol and 5 g of Tween 20® are emulsified in 90 g of a 10% strength aqueous solution of hydroxypropyl beta-cyclodextrin by stirring.

### EXAMPLE 10a

0.25 g of geranium oil is dissolved in 99.75 g of a 10% strength aqueous solution of hydroxypropyl beta-cyclodextrin by stirring.

### EXAMPLE 10b

1 g of geranium oil and 1 g of Tween 20® are emulsified in 98 g of a 10% strength aqueous solution of hydroxypropyl beta-cyclodextrin.

In the following examples, in each case the coating on nonwoven fabric with a stable emulsion and also a stable aqueous solution with the maximum amount of odorous substance comprising the odorous substance specified in each case is described. a) and c) each describes the prior art and b) the coating with a composition according to the invention.

### EXAMPLE 11

10 g of the stable solution from example 3a or 10 g of the stable aqueous emulsions from example 3b are applied to a piece of nonwoven of size 10 cm×10 cm (weight 5 g) by spraying. These nonwoven samples are dried and stored at room temperature. The analytical content determination of the guest is carried out by means of SPME analysis.

For the SPME analysis, in each case 180 mg of coated nonwoven are measured after 0, 3, 7 and 30 days. FIG. 3 shows the volatile constituents in the gas space.

### EXAMPLE 12

10 g of the stable solution from example 5a or 10 g of the stable aqueous emulsions from example 5b are applied to a piece of nonwoven of size 10 cm×10 cm (weight 5 g) by spraying. These nonwoven samples are dried and stored at room temperature. The analytical content determination of the guest is carried out by means of SPME analysis.

For the SPME analysis, in each case 180 mg of coated nonwoven are measured after 0, 3, 7 and 30 days. FIG. 4 shows the volatile constituents in the gas space.

The sensory determination of the odor strength of the nonwoven samples prepared in example 11 and example 12 takes place at the start and after storage for 3, 4, 7 and 9 weeks in daylight and at room temperature. The results for example 11 are shown in FIG. 5 and the results for example 12 are shown in FIG. 6.

In the following example, the coating of a wall with a solvent-free interior wall paint based on water comprising a stable emulsion according to the invention or a stable aqueous solution with the maximum amount of odorous substance (prior art) is described. The emulsion according to the invention or the stable aqueous solution comprises the odorous substance specified in each case in the respective maximum amount which still permits a stable emulsion/solution.

### EXAMPLE 13

18 g of solvent-free interior wall paint based on water are homogenized with 2 g of a stable emulsion according to example 7b or with 2 g of a stable aqueous solution according to example 7a or 7d). In each case 5 g of these solvent-free interior wall paints are applied to a piece of polyethylene (PE) film of size 20 cm×10 cm, dried and stored in daylight and at room temperature. The analytical content determination of the guest takes place by means of SPME analysis.

For the SPME analysis, in each case 500 mg of the wall paint are removed from the film and measured after 0, 5, 30 days.
FIG. 7 shows the volatile constituents in the gas space following the addition of 100 mg of water.

### EXAMPLE 14

### Preparation of a Gel for the Treatment of Acne of the Skin:

88 g of an emulsion of aqueous hydroxypropyl beta-cyclodextrin/TWEEN 20®/TEA TREE OIL (5% by weight tea tree oil in water) are mixed with 7 g of Wacker Belsil PDM 20®, 2 g of alpha-cyclodextrin-linoleic acid complex, 2 g of beta-cyclodextrin d-alpha-tocopherol complex and 1 g of Carbopol Ultrez 10 and stirred at room temperature for 30 minutes. The formulation is then neutralized with NaOH to a pH of 6-7, with formation of a gel.

### EXAMPLE 15

Preparation of a Gel with Repellent Effect on the Skin:

91.9 g of an emulsion of aqueous hydroxypropyl beta-cyclodextrin/TWEEN 20®/with 5% by weight geraniol are mixed with 7 g of Wacker Belsil PDM 20®, 1 g of Carbopol® Ultrez 10 (polyacrylic acid) and 0.1 g of Kathon® CG (substance mixture of fungicides, bactericides and algicides based on 5-chloro-2-methyl-, 2-methyl- or 2-octylisothiazol-3-one) and stirred at room temperature for 30 minutes. The formulation is then neutralized with NaOH to a pH of 6-7, with formation of a gel.

1.-9. (canceled)

10. An aqueous emulsion comprising a cyclodextrin derivative, a hydrophobic fragrance and a polysorbate, wherein the cyclodextrin derivative is present in an amount of from 6 to 35% by weight, the hydrophobic fragrance in an amount of from 3 to 20% by weight, and the polysorbate in an amount of from 4 to 20% by weight, and water.

11. The aqueous emulsion of claim 10, wherein the cyclodextrin derivative is present in an amount of from 10 to 20% by weight, the hydrophobic fragrance in an amount of from 6% to 12% by weight, and the polysorbate in an amount of from 6 to 12% by weight, and water.

12. The emulsion of claim 10, wherein the hydrophobic fragrance is a perfume.

13. The emulsion of claim 10, wherein the hydrophobic fragrance has an antimicrobial, fungicidal or, insect-repelling effect.

14. The emulsion of claim 11, wherein the hydrophobic fragrance has an antimicrobial, fungicidal or, insect-repelling effect.

15. The emulsion of claim 10, wherein the cyclodextrin derivative is selected from the group consisting of methyl beta-cyclodextrin, hydroxypropyl beta-cyclodextrin, and mixtures thereof.

16. The emulsion of claim 10, wherein the polysorbate is selected from the group consisting of polyoxyethylene(20) sorbitan monooleate, polyoxyethylene sorbitan tristearate, polyoxyethylene(20) sorbitan monolaurate, and mixtures thereof.

17. The emulsion of claim 10, wherein, besides water, the emulsion comprises 6 to 35% by weight of methyl beta-cyclodextrin, 3 to 20% by weight of a fragrance and 4 to 20% by weight of polyoxyethylene(20) sorbitan monolaurate.

18. A method for preparing an emulsion of claim 10, comprising agitating a mixture of 6 to 35% by weight of cyclodextrin derivative and 3 to 20% by weight of hydrophobic fragrance and 4 to 20% by weight of polysorbate in water to form a stable emulsion.

19. A method for preparing an emulsion of claim 10, comprising agitating a mixture of 10 to 20% by weight of cyclodextrin derivative and 6 to 12% by weight of hydrophobic fragrance and 6 to 12% by weight of polysorbate in water to form a stable emulsion.

20. A coating composition comprising an emulsion of claim 10.

21. A surface coated with an emulsion of claim 10.

22. A surface coated with the coating composition of claim 20.
